(19) United States
(12) Patent Application Publication
(10) Pub. No.: US 2009/0099206 A1
(43) Pub. Date: Apr. 16, 2009

(54) SOLID FORMS OF (S)-2-AMINO-3-(4-(2-AMINO-6-((R)-2,2,2-TRIFLUORO-1-(3'-METHOXYBIPHENYL-4-YL)ETHOXY)PYRIMIDIN-4-YL)PHENYL)PROPANOIC ACID AND METHODS OF THEIR USE

(76) Inventors: Shinya Iimura, Lawrenceville, NJ (US); Hui-Yin Li, Hockessin, DE (US); Qiuling Song, Newark, DE (US); Wenxue Wu, Princeton Junction, NJ (US); Matthew Mangzhu Zhao, Edison, NJ (US)

Correspondence Address: LEXICON PHARMACEUTICALS, INC., 8800 TECHNOLOGY FOREST PLACE, THE WOODLANDS, TX, 77381-1160, US

(21) Appl. No.: 12/246,735

(22) Filed: Oct. 7, 2008

Related U.S. Application Data
(60) Provisional application No. 60/978,303, filed on Oct. 8, 2007.

(51) Int. Cl. A61K 31/505; C07D 239/26; A61P 1/00

(52) U.S. Cl. 514/256; 544/335

(57) ABSTRACT

Solid forms of (S)-2-amino-3-(4-(2-amino-6-((R)-2,2,2-trifluoro-1-(3′-methoxybiphenyl-4-yl)ethoxy)pyrimidin-4-yl)phenyl)propanoic acid and salts thereof are disclosed.

This application claims priority to U.S. provisional application No. 60/978,303, filed Oct. 8, 2007, the entirety of which is incorporated herein by reference.

### 1. FIELD OF THE INVENTION

This invention relates to solid forms of (S)-2-amino-3-(4-(2-amino-6-((R)-2,2,2-trifluoro-1-(3′-methoxybiphenyl-4-yl)ethoxy)pyrimidin-4-yl)phenyl)propanoic acid and salts thereof.

### 2. BACKGROUND OF THE INVENTION

Different solid forms of the same compound can have substantially different properties. For example, the amorphous form of a drug may exhibit different dissolution characteristics and different bioavailability patterns than its crystalline form(s), properties which can affect how the drug must be administered to achieve optimal effect. Amorphous and crystalline forms of a drug may also have different handling properties (e.g., flowability, compressibility), dissolution rates, solubilities and stabilities, all of which can affect the manufacture of dosage forms. Consequently, access to multiple forms of a drug is desirable for a variety of reasons. Moreover, regulatory authorities (e.g., the U.S. Food and Drug Administration) may require the identification of all solid forms of a new drug substance before approving products containing it. A. Goho, Science News 166(8):122-123 (2004).

Compounds may exist in one or more crystalline forms, but the existence and characteristics of those forms cannot be predicted with any certainty. In addition, no standard procedure exists for the preparation of all possible polymorphic forms of a compound. And even after one polymorph has been identified, the existence and characteristics of other forms can only be determined by additional experimentation. Id.

### 3. SUMMARY OF THE INVENTION

This invention is directed, in part, to solid forms of the tryptophan hydroxylase inhibitor (S)-2-amino-3-(4-(2-amino-6-((R)-2,2,2-trifluoro-1-(3′-methoxybiphenyl-4-yl)ethoxy)pyrimidin-4-yl)phenyl)propanoic acid and pharmaceutically acceptable salts thereof. Particular solid forms are crystalline.

One embodiment of the invention encompasses pharmaceutical compositions comprising the solid forms described herein.

### 4. BRIEF DESCRIPTION OF THE FIGURES

Certain aspects of the invention may be understood with reference to the attached figures.

FIG. 1 is an X-ray diffraction pattern of a crystalline solid form of anhydrous (S)-2-amino-3-(4-(2-amino-6-((R)-2,2,2-trifluoro-1-(3′-methoxybiphenyl-4-yl)ethoxy)pyrimidin-4-yl)phenyl)propanoic acid tosylate. The diffractogram was obtained using a Rigaku MiniFlex diffractometer (Cu Kα radiation).

FIG. 2 is an X-ray diffraction pattern of a crystalline solid form of (S)-2-amino-3-(4-(2-amino-6-((R)-2,2,2-trifluoro-1-(3′-methoxybiphenyl-4-yl)ethoxy)pyrimidin-4-yl)phenyl)propanoic acid tosylate monohydrate. The diffractogram was obtained using a Bruker D8 Advance diffractometer (Cu Kα radiation).

FIG. 3 is an FT-Raman spectrum of a crystalline solid form of (S)-2-amino-3-(4-(2-amino-6-((R)-2,2,2-trifluoro-1-(3′-methoxybiphenyl-4-yl)ethoxy)pyrimidin-4-yl)phenyl)propanoic acid tosylate monohydrate. The spectrum was obtained using a Bruker RFS100 spectrometer (1064 nm excitation).

FIG. 4 is an X-ray diffraction pattern of a crystalline solid form of (S)-2-amino-3-(4-(2-amino-6-((R)-2,2,2-trifluoro-1-(3′-methoxybiphenyl-4-yl)ethoxy)pyrimidin-4-yl)phenyl)propanoic acid tosylate dihydrate. The diffractogram was obtained using a Rigaku MiniFlex diffractometer (Cu Kα radiation).

### 5. DETAILED DESCRIPTION OF THE INVENTION

This invention is directed, in part, to solid (e.g., crystalline) forms of (S)-2-amino-3-(4-(2-amino-6-((R)-2,2,2-trifluoro-1-(3′-methoxybiphenyl-4-yl)ethoxy)pyrimidin-4-yl)phenyl)propanoic acid, and pharmaceutically acceptable salts thereof. The compound is an inhibitor of tryptophan hydroxylase. When administered to animals, the compound decreases peripheral serotonin levels, and may be used to treat a wide range of diseases and disorders. See U.S. patent application Ser. Nos. 11/638,677, filed Dec. 12, 2006, and 60/946,246, filed Jun. 26, 2007.

This invention is also directed to dosage forms comprising solid forms of (S)-2-amino-3-(4-(2-amino-6-((R)-2,2,2-trifluoro-1-(3′-methoxybiphenyl-4-yl)ethoxy)pyrimidin-4-yl)phenyl)propanoic acid, and to methods of their use.

### 5.1. Definitions

Unless otherwise indicated, the phrases “disease or disorder mediated by peripheral serotonin” and “disease and disorder mediated by peripheral serotonin” mean a disease and/or disorder having one or more symptoms, the severity of which are affected by peripheral serotonin levels.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the terms “prevent,” “preventing” and “prevention” contemplate an action that occurs before a patient begins to suffer from the specified disease or disorder, which inhibits or reduces the severity of the disease or disorder. In other words, the terms encompass prophylaxis.

Unless otherwise indicated, a “prophylactically effective amount” of a compound is an amount sufficient to prevent a disease or condition, or one or more symptoms associated with the disease or condition, or to prevent its recurrence. A prophylactically effective amount of a compound means an amount of therapeutic agent, alone or in combination with other agents, which provides a prophylactic benefit in the prevention of the disease or condition. The term “prophylactically effective amount” can encompass an amount that improves overall prophylaxis or enhances the prophylactic efficacy of another prophylactic agent.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder or one or more of its symptoms, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should also be noted that any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. Structures that represent compounds with one or more chiral centers, but which do not indicate stereochemistry (e.g., with bolded or dashed lines), encompasses pure stereoisomers and mixtures (e.g., racemic mixtures) thereof. Similarly, names of compounds having one or more chiral centers that do not specify the stereochemistry of those centers encompass pure stereoisomers and mixtures thereof.

### 5.2. Solid Forms

This invention is directed to solid forms of (S)-2-amino-3-(4-(2-amino-6-((R)-2,2,2-trifluoro-1-(3′-methoxybiphenyl-4-yl)ethoxy)pyrimidin-4-yl)phenyl)propanoic acid:

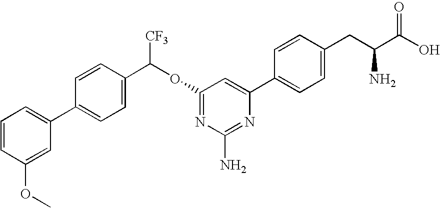

and salts thereof. Particular salts are crystalline. Specific salts include tosylate and maleate salts.

One embodiment of the invention encompasses anhydrous and hydrated crystalline forms of (S)-2-amino-3-(4-(2-amino-6-((R)-2,2,2-trifluoro-1-(3′-methoxybiphenyl-4-yl)ethoxy)pyrimidin-4-yl)phenyl)propanoate tosylate.

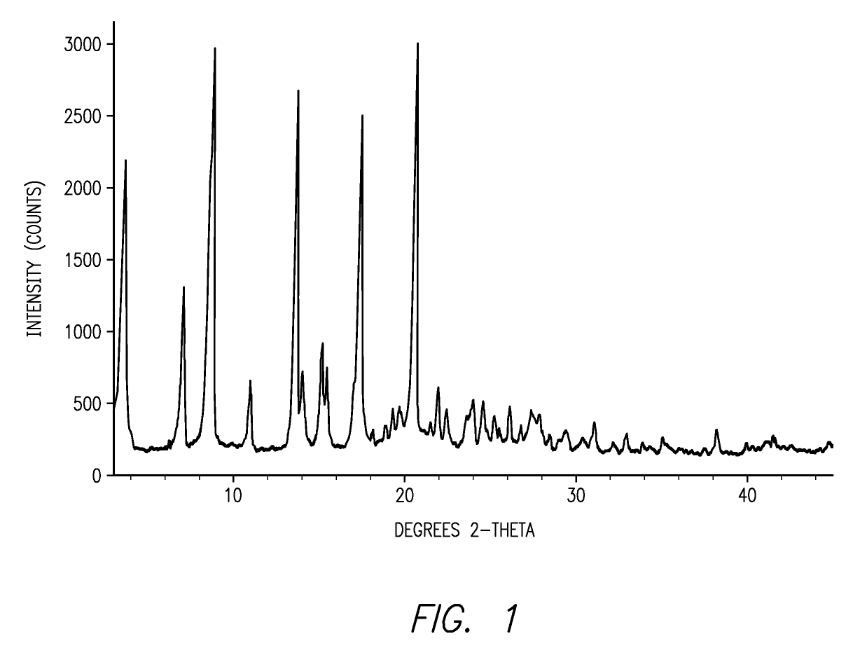

A particular form of this compound is (S)-2-amino-3-(4-(2-amino-6-((R)-2,2,2-trifluoro-1-(3′-methoxybiphenyl-4-yl)ethoxy)pyrimidin-4-yl)phenyl)propanoate tosylate anhydrate, having a melting point of about 241° C. as determined by DSC (onset temperature). In this context, the term “about” means±5.0° C. This form provides an X-ray diffraction (XRPD) pattern containing peaks at one or more of about 3.5, 7.0, 8.6, 10.9, 13.5, 14.0, 15.1, 17.3 and/or 20.5 degrees 2θ. In this context, the term “about” means±0.3 degrees. As those skilled in the art are well aware, the relative intensities of peaks in a XRPD pattern of a crystalline material can vary depending on how the sample is prepared and how the data is collected. With this in mind, an example of a XRPD pattern of this crystalline form is provided in FIG. 1.

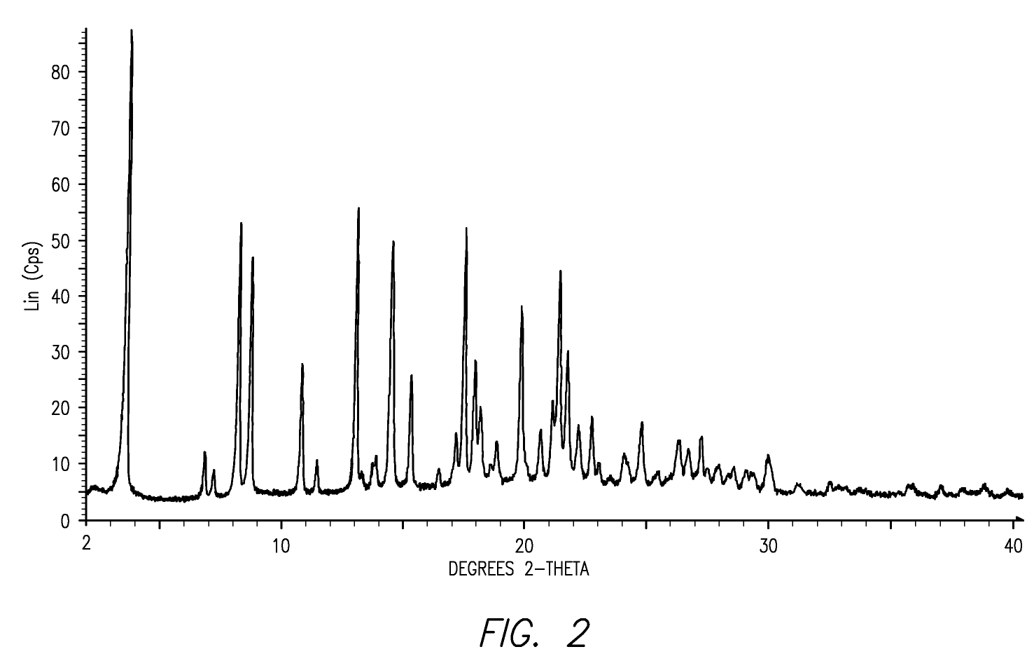

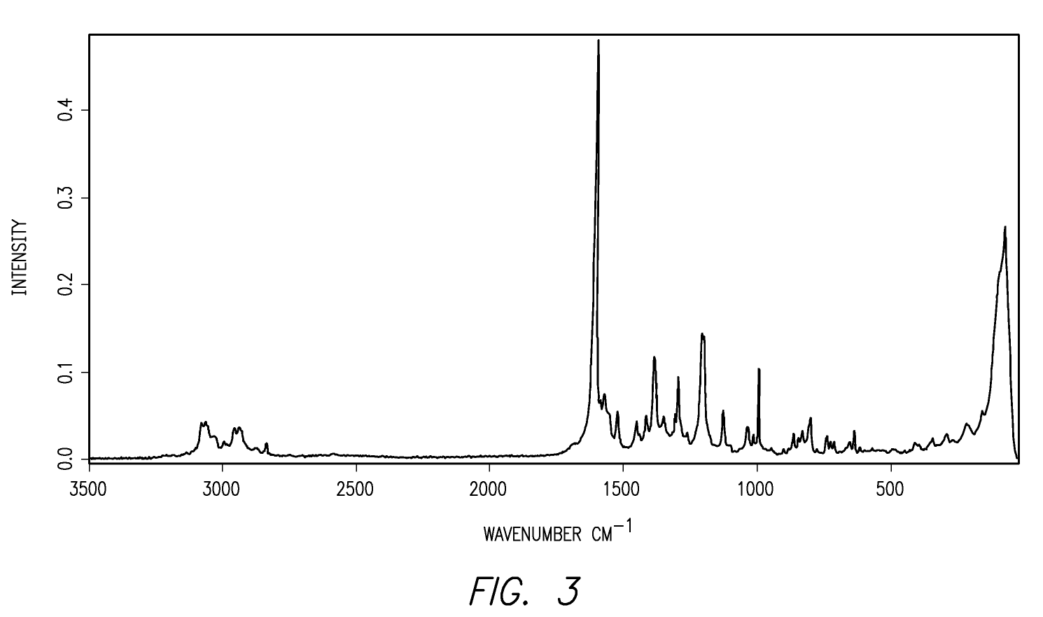

Another form of this compound is (S)-2-amino-3-(4-(2-amino-6-((R)-2,2,2-trifluoro-1-(3′-methoxybiphenyl-4-yl)ethoxy)pyrimidin-4-yl)phenyl)propanoate tosylate monohydrate having a melting point of about 221° C. as determined by DSC (onset of an endothermic peak having a maximum at about 227° C.). In this context, the term “about” means±5.0° C. This form provides an XRPD pattern containing peaks at one or more of about 3.6, 8.2, 8.7, 13.1, 14.5, 17.5, 18.0, 19.9 and/or 21.4 degrees 2θ. In this context, the term “about” means±0.3 degrees. An example of a XRPD pattern of this crystalline form is provided in FIG. 2. FIG. 3 provides an example of a Raman spectrum of this form.

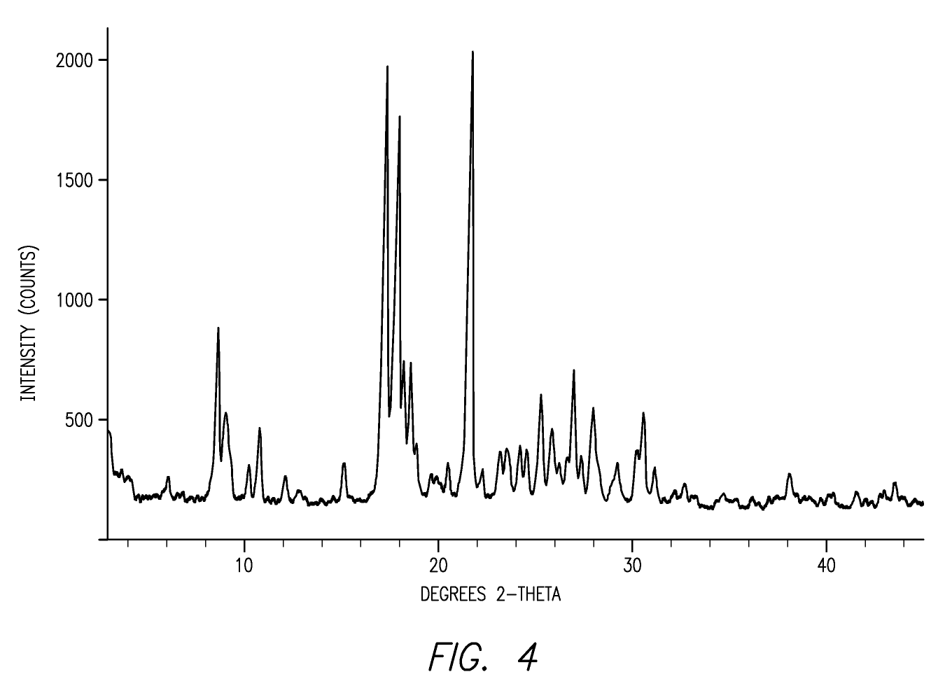

Another form of this compound is (S)-2-amino-3-(4-(2-amino-6-((R)-2,2,2-trifluoro-1-(3′-methoxybiphenyl-4-yl)ethoxy)pyrimidin-4-yl)phenyl)propanoate tosylate dihydrate having a melting point of about 238° C. as determined by DSC (onset of an endothermic peak having a maximum at about 242° C.). In this context, the term “about” means±5.0° C. This form provides an XRPD pattern containing peaks at one or more of about 8.6, 9.0, 17.2, 17.8, 18.6, 21.6, 25.2 and/or 26.9 degrees 2θ. In this context, the term “about” means±0.3 degrees. An example of an XRPD pattern of this form is provided in FIG. 4.

Another embodiment of this invention encompasses anhydrous and hydrated forms of (S)-2-amino-3-(4-(2-amino-6-((R)-2,2,2-trifluoro-1-(3′-methoxybiphenyl-4-yl)ethoxy)pyrimidin-4-yl)phenyl)propanoate maleate.

This invention encompasses solids that are mixtures of both amorphous and crystalline forms. Certain such solids comprise crystalline (S)-2-amino-3-(4-(2-amino-6-((R)-2,2,2-trifluoro-1-(3′ -methoxybiphenyl-4-yl)ethoxy)pyrimidin-4-yl)phenyl)propanoic acid or a pharmaceutically salt thereof in an amount of at least about 50, 75, 80, 85, 90, 95 or 99 weight percent.

Crystalline salts of (S)-2-amino-3-(4-(2-amino-6-((R)-2,2,2-trifluoro-1-(3′-methoxybiphenyl-4-yl)ethoxy)pyrimidin-4-yl)phenyl)propanoic acid can be prepared by heating a solution comprising the compound and a pharmaceutically acceptable acid, reducing the solubility of the resulting salt, and isolating the crystalline salt. In one embodiment, the solution is in THF/water. In a particular method, the THF/water solution is heated to about 40-60° C. Then, crystallization of the salt is effected by adding an anti-solvent (e.g., acetonitrile) to the hot solution, which is then allowed to cool.

### 5.3. Methods of Treatment

This invention encompasses a method of inhibiting tryptophan hydroxylase (TPH), which comprises contacting TPH with a compound of the invention (i.e., a compound disclosed herein). In a particular method, the TPH is the TPH1 isoform. In another, the TPH is the TPH2 isoform. In a particular method, the inhibition is in vitro. In another, the inhibition is in vivo.

This invention encompasses methods of treating, preventing and managing various diseases and disorders mediated by peripheral serotonin, which comprise inhibiting TPH1 activity in a patient in need of such treatment, prevention or management.

Particular diseases and disorders include carcinoid syndrome and gastrointestinal diseases and disorders. Examples of specific diseases and disorders include abdominal pain (e.g., associated with medullary carcinoma of the thyroid), anxiety, carcinoid syndrome, celiac disease, constipation (e.g., constipation having an iatrogenic cause, and idiopathic constipation), Crohn's disease, depression, diabetes, diarrhea (e.g., bile acid diarrhea, enterotoxin-induced secretory diarrhea, diarrhea having an iatrogenic cause, idiopathic diarrhea (e.g., idiopathic secretory diarrhea), and traveler's diarrhea), emesis, functional abdominal pain, functional dyspepsia, irritable bowel syndrome (IBS), lactose intolerance, MEN types I and II, Ogilvie's syndrome, Pancreatic Cholera Syndrome, pancreatic insufficiency, pheochromacytoma, scleroderma, somatization disorder, and Zollinger-Ellison Syndrome.

In particular methods of the invention, the treatment, management and/or prevention of a disease or disorder is achieved while avoiding adverse effects associated with alteration of central nervous system (CNS) serotonin levels. Examples of such adverse effects include agitation, anxiety disorders, depression, and sleep disorders (e.g., insomnia and sleep disturbance).

### 5.4. Pharmaceutical Compositions

This invention encompasses pharmaceutical compositions and dosage forms comprising solid form of the invention. Pharmaceutical compositions and dosage forms of this invention may optionally contain one or more pharmaceutically acceptable carriers or excipients. Certain pharmaceutical compositions are single unit dosage forms suitable for oral, topical, mucosal (e.g., nasal, pulmonary, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration may require enteric coatings to protect the active ingredient from degradation within the gastrointestinal tract. In another example, the active ingredient may be administered in a liposomal formulation to shield it from degradative enzymes, facilitate transport in circulatory system, and/or effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18^{th} ed., Mack Publishing, Easton Pa. (1990).

### 6. EXAMPLES

### 6.1. Preparation of (R)-1-(4-Bromophenyl)-2,2,2-trifluoroethanol

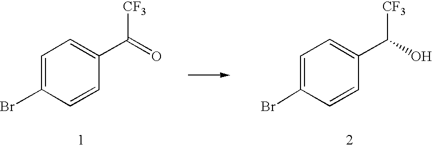

This compound was prepared based on a literature procedure (Ohkuma, et al. J. Am. Chem. Soc., 120:13529-13530 (1998)). To a 1 L high pressure vessel was charged 4-bromo-trifluoroacetophenone (1, Wilmington PharmaTech, Delaware, 100.0 g, 395 mmol), potassium tert-butoxide (1 M solution in 2-methyl-2-propanol, 5.0 ml, 10.0 mmol, 0.025 eq), and catalyst [(trans)-RuCl₂[(R)-Xyl-P-Phos][(R)-DIAPEN] (Johnson Matthey, New Jersey, 200 mg, 0.16 mmol, 0.04% mol). The mixture was dissolved in anhydrous 2-propanol (175 ml) and the entire vessel was purged with argon by 3 vacuum-thaw cycles. The reaction mixture was then purged with hydrogen by 3 vacuum-thaw cycles. The reaction was carried out under 60 psi hydrogen atmosphere. After 24 hours of stirring and no more hydrogen consumption, the reaction was deemed complete by GC-MS analysis (no more starting ketone). The contents of the reaction vessel were transferred to a round bottom flask with MeOH rinsing (3'20 ml), and concentrated under reduced pressure until no more solvent was distilling off. The resulting orange-brown oil was then dissolved in heptane (1000 ml) and washed with water (2×100 ml), brine (100 ml) and dried over sodium sulfate. To the dried organic layer was added Darco® activated charcoal (20 g) and Hyflo® Super Cel (20 g) and the mixture was heated at 70° C. for 1 hours. The mixture was filtered hot to give a light yellow solution. The filtrate was concentrated under reduced pressure with heating (˜50-60° C.) until no more solvent was distilling. The resulting yellow oil was dissolved in 60° C. warm heptane (350 ml) and allowed to stir while cooling. As the temperature cooled to room temperature, white solid began to precipitate. After 4 hours of stirring, the solids were filtered and dried to give the titled product (63.5 g, 63%, >99% ee) as a white powder. m.p.: 56.7° C. [α]=−30.1 (c1.09, ethanol). GC-MS (CT): MH⁺=255.8. ¹H NMR (CDCl₃) δ 7.58 (m, 2H), 7.42 (d, J=8.3 Hz, 2H), 5.00 (m, 1H), 2.62 (d, J=4.3 Hz, 1H). ¹³CNMR (CDCl₃): δ 133.2, 132.2, 129.5, 125.7, 124.3 (q, J=282 Hz), 72.6 (q, J=32 Hz). ¹⁹F NMR (CDCl₃): δ −78.5 (d, J=5.6 Hz).

### 6.2. Preparation of (S)-1-(4-Bromophenyl)-2,2,2-trifluoroethanol

Using a procedure similar to the above example, the title compound was prepared using catalyst [(trans)-RuCl₂[(S)-Xyl-P-Phos][(S)-DIAPEN] (Johnson Matthey, New Jersey).

### 6.3. Preparation of (R)-2,2,2-Trifluoro-1-(p-tolyl)ethanol

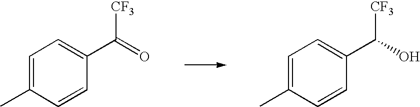

Similarly, 2,2,2,-trifluoro-1-(p-tolyl)ethanone was hydrogenated using catalyst [(trans)-RuCl₂[(R)-Xyl-P-Phos][(R)-DIAPEN] to give the title compound. m.p.: 44.2° C. ¹H NMR (CDCl₃): δ 7.38 (d, J=6.0 Hz, 2H), 7.25 (d, J=6.0 Hz, 2H), 5.00 (dq, J₁=6.6 Hz, J₂=3.3 Hz, 1H), 2.49 (d, J=3.8 Hz, 1H), 2.42 (s, 3H).

### 6.4. Preparation of (S)-2,2,2-Trifluoro-1-(p-tolyl)ethanol

Similarly, the title compound was prepared using catalyst [(trans)-RuCl₂[(S)-Xyl-P-Phos][(S)-DIAPEN].

### 6.5. Preparation of (R)-2,2,2-Trifluoro-1-(3′-methoxybiphenyl-4-yl)ethanol

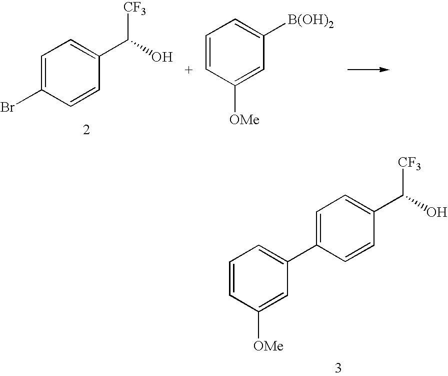

To a stirred solution of (R)-1-(4-bromophenyl)-2,2,2-trifluoroethanol (2, 69 g, 0.27 mol, >99% ee), 3-methoxy phenylboronic acid (Matrix, 51 g, 0.34 mol, 97% purity), and bis(triphenylphosphine)palladium(II) dichloride (0.95 g, 0.5% mol) in ethanol (560 ml) was added a solution of potassium carbonate (112 g, 0.81 mol) in water (140 ml) under nitrogen. The resulting mixture was heated at 75° C. for 1 hour and deemed complete by GC-MS or TLC. After reaction mixture was cooled to 40° C., it was filtered through a pad of Celite, washed with methanol (3×100 ml). The filtrate was diluted with 100 ml of water and concentrated. The resulting syrup was dissolved in 700 ml of ethyl acetate and washed with 1 N sodium hydroxide (2×100 ml), water (2×100 ml) and brine (1×100 ml). The organic layer was heated with activated carbon (14 g) and Hyflo Super Cel (14 g) at 60° C. for 1 hours. This mixture was filtered hot and washed with ethyl acetate (100 ml) and then concentrated to a syrup. This syrup was immediately dissolved in 1% ethyl acetate/heptane (700 ml) and stirred for 4 hours. The resulting slurry was filtered and dried to give the title compound as a white crystalline solid (3, 68 g, 89% yield, >99% ee).

In an alternative crystallization method, the crude product syrup/solid (10 g) was dissolved in MTBE (10 ml) and diluted with heptane (200 ml). The solution was concentrated to about 70 ml under reduced pressure. This mixture was stirred at room temperature overnight and the resulting slurry was filtered and dried to give the title compound (3, 8.8 g) as a white crystalline solid. m.p.:107.6° C. [α]=−31.85 (c 1.067, ethanol). LC-MS (ESI): MH⁺=283.1. ¹H NMR (CDCl₃): δ 7.66 (m, 2H), 7.56 (d, J=8.2 Hz, 2H), 7.42 (t, J=7.8 Hz, 2H), 7.20 (m, 1H), 7.14 (m, 1H), 6.95 (m, 1H), 5.82 (q, J=6.6 Hz, 1H), 3.85 (s, 3H), 2.63 (br s, 1H). ¹³C NMR (CDCl₃): δ 160.3, 142.6, 142.2, 133.5, 130.3, 128.3, 127.8, 124.8 (q, J=282 Hz), 120.1, 113.4, 113.3, 73.0 (q, J=32 Hz), 55.7. ¹⁹F NMR (CDCl₃): δ −78.3 (d, J=6.4 Hz). Residual palladium: 11 ppm. Anal. Calcd for C₁₅H₁₃F₃O₂: C, 63.83; H, 4.64. Found: C, 63.78; H, 4.60.

### 6.6. Preparation of (R)-2,2,2-Trifluoro-1-(3′-methoxybiphenyl-4-yl)ethanol

A 22-L, round-bottom flask equipped with a mechanical stirrer, a thermocouple attached to a temperature controller, and a condenser with a nitrogen line was charged with compound 2 (1.00 kg, 1 wt, 3.92 mol) and ethanol (4.5 L, 4.5 vol). The mixture was sparged with nitrogen for 10 minutes and (Ph₃P)₂PdCl₂ (12.6 g, 0.0126 wt, Strem) was added. Following additional sparging with nitrogen, a solution of K₂CO₃ (1.63 kg, 3 equiv) in water (2 vol) was added. The mixture was heated to 75° C. under nitrogen and then approximately 20% of a solution of 3-methoxy phenylboronic acid (715 g, 4.70 mol, 1.2 equiv, Usun) in ethanol (4.5 vol) was added via a peristaltic pump. After 20 minutes, an in-process control (IPC) sample was taken and showed that the boronic acid had been consumed. This process was repeated until all of the boronic acid was added. After stirring for a further 20 minutes, HPLC analysis showed that the reaction was complete. The heat was switched off and at 69° C., water (3.6 vol) was added. The reaction mixture was then filtered at 50° C. through a pad of celite (Celpure P300, 0.15 wt., Sigma) and the filter cake was washed with methanol (2×2.5 vol). The filtrate was concentrated under reduced pressure at 40-45° C. to 5 vol. The slurry was then transferred to a separatory funnel and MTBE (10 vol) was added. The mixture was then washed with a 50% solution of sodium hydroxide (0.6 vol). After stirring, the layers were separated and the aqueous phase was extracted with MTBE (1.5 vol). The organic extracts were combined and washed with water (1 vol) followed by 20% aqueous sodium chloride (1 vol) to provide 11.9 volumes of organic product solution. The solution was transferred to a reactor, treated with a slurry of Darco G-60 (0.3 wt) in MTBE (1 vol) and heated to 50° C. After 90 minutes, the mixture was filtered through a pad of Celpure P300 (0.15 wt) and washed with MTBE (2×3 vol).

The filtrate (14.8 vol) was transferred to a reactor and distilled under vacuum at 45° C. to remove MTBE. The filtrate was reduced to 6.7 volumes over 2.5 hours and then heptane (3.15 vol) was added. The solution was further distilled at 50° C. to 6.7 vol over 1 hour and then additional heptane (3.15 vol) was added. The solution was concentrated to 6.7 vol at 55° C. over 1.5 hours and then heptane was added (3.15 vol). Precipitation was observed immediately and the distillation was continued under vacuum at 60° C. After 2.5 hours, the distillation was stopped (7 vol remaining), the heat was switched off and the batch was cooled overnight to ambient temperature. The batch was filtered at 24° C. and washed with heptane (1.5 vol). The solids were dried at room temperature under vacuum over the weekend to provide 799.7 g of 3 as a white solid [72% yield, >99% (AUC)].

### 6.7. Preparation of (R)-2,2,2-Trifluoro-1-(3′-fluorobiphenyl-4-yl)ethanol

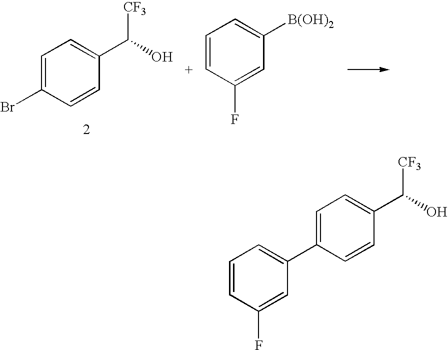

Similar to the above procedure, the title compound was prepared from (R)-1-(4-bromophenyl)-2,2,2-trifluoroethanol (2) and 3-fluorophenylboronic acid. ¹H NMR (CDCl₃): δ 7.62(d, J=6.0 Hz, 2H), 7.56 (d, J=6.3 Hz, 2H), 7.42 (m, 2H), 7.28 (m, 1H), 7.06 (m, 1H), 5.82 (q, J=5.1 Hz, 1H).

### 6.8. Preparation of (S)-Methyl 2-(tert-butoxycarbonylamino)-3-(4-(trifluoromethylsulfonyloxy)phenyl)propanoate

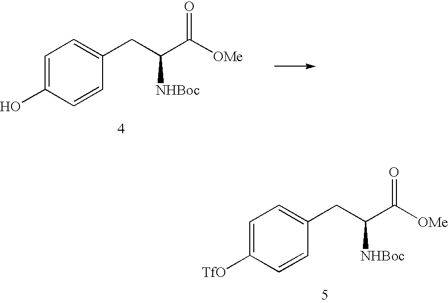

This compound was prepared based on a literature procedure (Shieh, et al. J. Org. Chem., 1992, 57, 379-381). To a solution of Boc-Tyr-OMe (4, Bachem, California, 100 g, 0.34 mol) and N-methylmorpholine (51 g, 1.5 eq) in dichloromethane (1000 ml) was added triflic anhydride (100 g, 1.05 eq) over 2 hours at −5 to −15° C. The resulting red solution was stirred at −10° C. for 10 minutes. HPLC analysis showed complete disappearance of starting material. The reaction was quenched with 10% citric acid (500 ml). The organic layer was washed with 10% citric acid (500 ml) followed by water (500 ml). The resulting light pink solution was concentrated under reduced pressure to 200 ml. This was diluted with acetonitrile (600 ml) and further concentrated to a 200 g solution. This solution was used in the next step without further purification. Estimated yield was 98% by stripping a sample to dryness to give a low melting pale yellow solid. LC-MS (ESI): MH⁺=428.0, MNH₄⁺445.0. ¹H NMR (CDCl₃) δ 7.16 (m, 4H), 4.95 (d, J=7.1 Hz, 1H), 4.53 (m, 1H), 3.64 (s, 3H), 3.10 (dd, J₁=5.7 Hz, J₂=13.8 Hz, 1H), 2.97 (dd, J₁=6.3 Hz, J₂=13.6 Hz, 1H), 1.34 (s, 9H). ¹³C NMR (CDCl₃) δ 172.3, 155.4, 149.0, 137.4, 131.5, 121.7, 119.1 (q, J=321 Hz), 80.54, 54.62, 52.7, 38.3, 28.6. ¹⁹F NMR (CDCl₃) δ −73.4.

### 6.9. Preparation of (S)-2-(Tert-butoxycarbonylamino)-3-(4-(4,4,5,5-tetramethyl-1,3,2-dioxaborolan-2-yl)phenyl)propanoic Acid

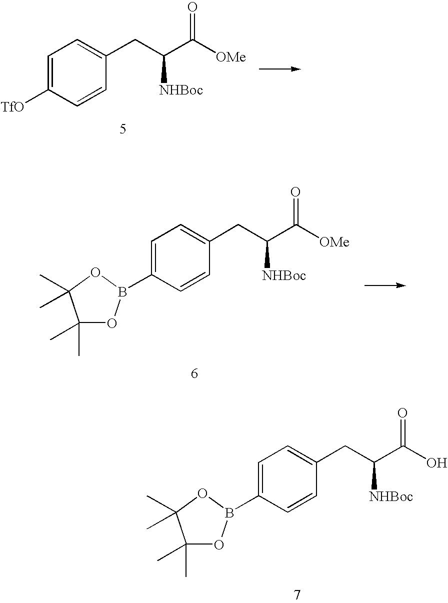

This ester compound 6 was prepared based on a literature procedure (Firooznia, et al., Tetrahedron Lett., 1999, 40, 213-216). Bis(pinacolato)diboron (90 g, 1.1 eq), potassium acetate (63 g, 2 eq), tricyclohexylphosphine (2.3 g, 2.5% mol), and palladium acetate (0.72 g, 1 mol %) were mixed in acetonitrile (950 ml) and the resulting mixture stirred at room temperature for 5 minutes. The above triflate (5) solution (190 g, 0.32 mol) was added and the resulting mixture was heated at 80° C. for 1 hour and cooled. HPLC showed complete consumption of the starting material. The reaction mixture was quenched with aqueous potassium bicarbonate solution (57 g in 475 ml water) and resulting mixture was stirred at room temperature for 30 minutes. The mixture was filtered through a pad of 20μ cellulose to remove palladium black. A sample of the organic layer was concentrated and purified by column chromatography (gradient: 1:10 to 1:4 ethyl acetate/hexanes) to give the ester compound 6 as a clear oil. LC-MS (ESI): MH⁺=406.2, MNH₄⁺=423.2, M₂H⁺=811.5, M₂NH₄⁺=428.5. ¹H NMR (CDCl₃) δ 7.76 (d, J=8.1 Hz, 2H), 7.15 (d, J=7.6 Hz, 2H), 4.96 (d, J=7.3 Hz, 1H), 4.60 (m, 1H), 3.72 (s, 3H), 3.13 (m, 2H), 1.44 (s, 9H), 1.36 (s, 12H).

The above organic layer of 6 was stirred with aqueous lithium hydroxide solution (23 g in 500 ml water) at room temperature for 30 minutes. The pH of the resulting slurry was adjusted to about 10 with 6 N hydrochloric acid and filtered. The cake was washed with water (200 ml). Acetonitrile was removed from the filtrate under reduced pressure to give an aqueous slurry (950 ml, additional water was added during distillation). The slurry was filtered through a pad of 20 μm cellulose and washed with water (200 ml). The filtrate was washed with MTBE (500 ml) and rediluted with 700 ml MTBE. The mixture was acidified to pH about 4.5 with 6 N hydrochloric acid. The organic layer was washed with water (500 ml) and concentrated under reduced pressure to the titled product (7) as a brown oil (206 g, 95% yield based on estimated purity by NMR). The crude product was used directly in the following step. LC-MS (ESI): MH⁺=392.2, MNH₄⁺=409.2, M₂H⁺=783.4, M₂NH₄⁺=800.4. ¹H NMR (CDCl₃) δ 7.95 (br s, 1H), 7.76 (d, J=7.8 Hz, 2H), 7.21 (d, J=7.6 Hz, 2H), 5.03 (d, J=7.8 Hz, 1H), 4.62 (m, 1H), 3.18 (m, 2H), 1.43 (s, 9H), 1.35 (s, 12H). ¹³C NMR (CDCl₃) δ 175.8, 155.7, 139.7, 135.4, 129.2, 84.2, 80.5, 54.5, 38.3, 28.7, 25.2.

Compound 7 was also isolated by crystallization. For example, the above MTBE solution of 7 was dried with anhydrous Na₂SO₄ and concentrated to about 1.0 vol under vacuum. Heptane (2.5 vol) was added and concentrated to about 1.5 vol under vacuum. Heptane (4.2 vol) was added slowly at 36˜42° C. followed by cooling slowly to 5˜10° C. The resulting slurry is filtered, washed by heptane, and dried under vacuum at 20-30° C. to give the product 7 in about 76% yield.

### 6.10. Alternative Crystallization of (S)-2-(Tert-butoxycarbonylamino)-3-(4-(4,4,5,5-tetramethyl-1,3,2-dioxaborolan-2-yl)phenyl)propanoic Acid

A 1 L jacketed three-necked round bottom flask with mechanical stirrer, rubber septum with temperature probe, and gas bubbler was charged with 100 ml of an ethanol solution containing 50.88 g 7. The solution was set stirring under nitrogen, diluted with 35 ml ethanol, then with 50 ml 2-propanol, and was heated to ˜60° C. Then, 250 ml water were added to reach the cloudy point and the turbid solution was held at ˜60° C. for 75 minutes followed by cooling to ˜10° C. over ˜1.5 hours. After 45 minutes, the mixture was biphasic and was diluted with an additional 30 ml 2-propanol. The mixture was stirred under nitrogen at 10° C. overnight and the resulting white fine suspension was filtered. The collected solids were washed with 100 ml 9:1 water:2-propanol and were dried in vacuo at ˜50-60° C. to give 39.88 g 7 as a chalky white powder (78% recovery). The solid was in the filtrate was filtered and dried to afford 4.51 g of a pale yellow granular solid. HPLC suggested this material was mostly the boronic acid.

### 6.11. Preparation of (S)-3-(4-(2-Amino-6-chloropyrimidin-4-yl)phenyl)-2-(tert-butoxycarbonylamino)propanoic Acid

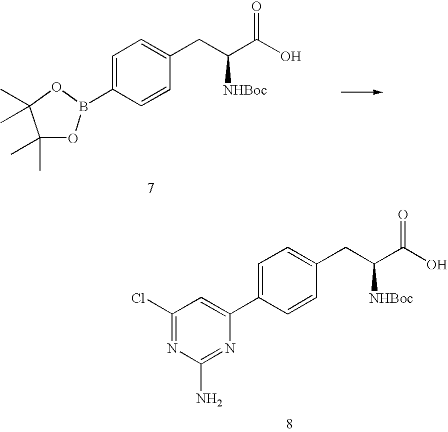

The above crude compound 7 (0.32 mol) was dissolved in ethanol (800 ml) and resulting solution was concentrated under reduced pressure to about 700 ml and diluted with ethanol (1300 ml). To this solution was added 2-amino-4,6-dichloropyrimidine (74 g, 1.4 eq), bis(triphenylphosphine)palladium(II) dichloride (2.3 g, 1 mol %), and aqueous potassium bicarbonate solution (97 g, 3 eq, 380 ml water). This mixture was heated at 75-80° C. for 2 hours, at which time HPLC analysis showed complete consumption of the starting material. Ethanol was removed from the filtrate under reduced pressure to give an aqueous slurry (600 ml, additional water was added during distillation). The slurry was filtered and washed with 200 ml water. The cake was dried at 50° C. under vacuum to give recovered 2-amino-4,6-dichloropyrimidine as a tan solid (30 g, 41% of original charge). ¹H NMR (DMSO-d₆) δ 7.58 (br s, 2H), 6.84 (s, 1H). ¹³C NMR (DMSO-d₆) δ 162.8, 160.9, 107.5. The filtrate was washed with ethyl acetate (400 ml) and diluted with 3:1 THF/MTBE (600 ml). The mixture was acidified to pH about 3.5. The organic layer was washed with brine (300 ml) and concentrated to give the crude product 8 as a red oil (180 g). This oil was redissolved in THF (300 ml), polish-filtered, and washed with THF (100 ml). The filtrate was diluted with isopropanol (400 ml) and the mixture was distilled atmospherically to about 300 ml. More isopropanol (400 ml) was added and distillation continued until the volume reached about 500 ml. The mixture was then cooled over 1 hour to 45° C. and held for 2 hours before it was cooled to room temperature over 1 hours. After an additional hour, the slurry was filtered, washed with isopropanol (150 ml), and dried at 50° C. under vacuum to give the product 8 as a light pink solid (46.2 g, 37% yield from Boc-Tyr-OMe, 4). Purity: 93.4% by HPLC. Chiral purity: >99% ee. Chiral analysis was performed on the corresponding methyl ester derivative, which was prepared using trimethylsilyldiazomethane. An analytical pure sample was obtained by column chromatography (gradient 1:20 to 1:10 methanol/dichloromethane). LC-MS (ESI) MH⁺=393.1, MH⁺+acetonitrile=434.1, M₂H⁺=785.3. ¹H NMR (DMSO-d₆) δ 12.60 (s, 1H), 8.02 (d, J=8.3 Hz, 2H), 7.38 (d, J=8.1 Hz, 2H), 7.23 (s, 1H), 7.13 (br s, 2H), 3.09 (dd, J₁=4.4 Hz, J₂=13.5 Hz, 1H), 2.91 (dd, J₂=10.5 Hz, J₂=13.8 Hz, 1H), 1.32 (s, 9H). ¹³C NMR (DMSO-d₆) δ 173.4, 165.8, 163.5, 161.0, 155.4, 141.4, 134.0, 129.4, 126.8, 104.4, 78.0, 54.8, 36.2, 28.1. Anal. Calcd for C₁₈H₂₁ClN₄O₄: C, 55.03; H, 5.39; N, 14.26. Found: C, 54.76; H, 5.65; N, 14.09.

HPLC analysis of the above mother liquor against an standard solution of compound 8 showed additional 38 g product 8 (30% yield from Boc-Tyr-OMe, 4). Product 8 was also partially recovered by further concentration of the mother liquor to give a total yield of 60% from Boc-Tyr-OMe, 4.

### 6.12. Preparation of (S)-3-(4-(2-Amino-6-chloropyrimidin-4-yl)phenyl)-2-(tert-butoxycarbonylamino)propanoic Acid

A 22-L, round-bottom flask equipped with a mechanical stirrer, a thermocouple attached to a temperature controller, and a condenser with a nitrogen line was charged with compound 7 (850 g, 1 wt, 2.17 mol), 2-amino-4,6-dichloropyrimidine (712.3 g, 2 equiv, Usun), and ethanol (13.6 L, 16 vol). The slurry was sparged with nitrogen for 10 min; then (Ph₃)₂PdCl₂ (18.3 g, 0.021 wt, Strem) was added and nitrogen sparging was continued for 10 minutes. A solution of potassium bicarbonate (783 g, 3.6 equiv) in water (3.2 L, 3.7 vol) was then charged to the reactor whereupon gas evolution was observed. The mixture was heated to 75° C. for a total of 11.5 hours and then cooled to 45° C. overnight. HPLC analysis after 9.5 h at 75° C. indicated that there were about 3.0% of 7 remaining (by conversion). The reaction was cooled to 45° C. and stirred overnight whereupon HPLC analysis indicated that there was <1.0% of 7 remaining.

The batch was then distilled under reduced pressure at 45° C. over a period of 15 hours to afford 4-5 L of a yellow slurry. The batch was then allowed to cool overnight. Water was added (3 vol) and after heating to 45° C., distillation was continued for 1 hours until no more distillate was collected. The vacuum was released and water (3 vol) was added to the batch. After allowing to settle, the batch was filtered through a slurry of cellulose powder (20 micron, 0.2 wt.) in water (1 vol). Water (2 vol) was added to the remaining solids/slurry in the reactor and this was filtered through a sintered glass funnel. This filtrate was then further filtered through the cellulose pad to afforded 11.2 L of product solution (13.2 vol).

The solution was then transferred to a separatory funnel containing EtOAc (3.3 vol). After stirring and separating, the aqueous phase was transferred to a 22-L reactor and then a solution of PBu₃ (212 ml, 0.25 vol, 97%) in EtOAc (3.5 vol) was charged to the reactor. The solution was heated at 50° C. for 2.5 hours. Additional EtOAc (3.3 vol) was added to the reactor and the contents were charged to a separatory funnel and the two phases separated. The aqueous phase (41° C.) was charged back to the separatory funnel and washed with additional EtOAc (3.3 vol). The two phases were separated and then the aqueous phase was charged to a 22-L reactor and heated to 45° C. Heptane (5 vol) was added to the reactor, the contents of the reactor were transferred to a separatory funnel and the two phases were separated. The aqueous phase (11.2 L, 13.2 vol) was charged to the 22-L reactor, diluted to 14 vol with water and then a slurry of Darco G-60 (0.2 wt) in water (1 vol) was charged to the reactor. The mixture was heated to 60° C. and stirred at 60° C. for 2 hours. The heat was switched off and the batch was stirred over the weekend. The batch was filtered through a pad of Celpure P300 (0.2 wt, Sigma) and washed with water (2×1.2 vol).

A 22-L, round-bottom flask equipped with a mechanical stirrer, a thermocouple attached to a temperature controller, and pH probe attached to a pH meter was charged with citric acid (127.5 g, 0.15 wt) and water (2 vol). The solution was heated to 40° C. and the pH of the solution was adjusted to 4.0 with a 2 M solution of sodium hydroxide. A solution of citric acid (40 wt %, 2 L) was charged to an addition funnel and was attached to the reactor. The basic solution of 8 was then transferred via peristaltic pump through an in-line filter to the citric acid solution and the pH was maintained at pH 4.0 with the 40% citric acid solution. Once the addition was complete, the batch was heated to 60° C. and stirred for 2 hours. The batch was then cooled overnight and the solids were filtered at 29° C. The cake was washed with water (2×2.5 vol) and then dried at 45-50° C. for 24 hours to provide 720 g of 8 (84% yield) with a purity of 85.9% (AUC).

### 6.13. Purification of (S)-3-(4-(2-Amino-6-chloropyrimidin-4-yl)phenyl)-2-(tert-butoxycarbonylamino)propanoic Acid

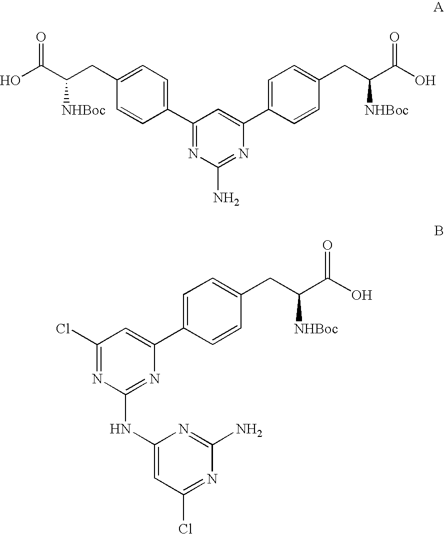

When prepared as described above, the captioned compound 8 typically contains about 6% of the diacid impurity A and about 4% amination product B. While compound 8 can be used in its crude form, it can be purified using the approaches described below.

Method 1. To a 3-necked 250 ml RB flask was added crude 8 (10.0 g, 25.4 mmol, 90% pure, with 6% A and 4% B), i-PrOH/toluene (1:1, 80 ml/80 ml, 8x/8x) and tert-butylamine (13.4 ml, 5.0 equiv). The resulting mixture was stirred and heated at 78° C. for 1 hour and then slowly cooled to 0° C., and stirred for another hour. The solids were collected by filtration and the cake was washed with 20 ml of i-PrOH/toluene (1:3). The cake was dried under vacuum to constant weight to provide the desired tert-butylamine salt of 8 as a pale yellow solid (8.8 g, 74% yield, 94% pure, 3% A, 3% B).

To a 3-necked 250 ml RB flask was added the tert-butylamine salt of 8 (20.0 g, 42.9 mmol) and followed by H₂O/THF/toluene (400 ml/200 ml/160 ml, 20x/10x/8x). The resulting mixture was heated to 60° C. and slowly added 6M HCl until pH of the mixture reached 4.0. The mixture was cooled to room temperature and the organic layer was separated. The organic layer was washed with H₂O (100 ml, 5x) and concentrated by rotary evaporating to around 160 ml of overall volume. The solids were collected by filtration and the cake was washed with 20 ml of toluene. The cake was dried under vacuum to constant weight to provide 8 as a pale yellow solid (15.0 g, 89% yield, 94% pure, 3% A, 3% B).

Method 2. To a 3-necked 250 ml RB flask was added crude 8 (20.0 g, 42.9 mmol, 90% pure, with 6% A and 4% B) and followed by THF/toluene (200 ml/160 ml, 10x/8x). The resulting mixture was heated to 60° C. for 1 hour and cooled to room temperature. THF was removed by rotary evaporating to around 160 ml of overall volume. The solids were collected by filtration and the cake was washed with 20 ml of toluene. The cake was dried under vacuum to constant weight to provide 8 as a pale yellow solid (11.8 g, 70% yield, 92.8% pure, 6.0% A, 1.3% B).

To a 3-necked 250 ml RB flask was added the above 8 (10.0 g, 25.4 mmol) and tert-butylamine (13.4 ml, 5 equiv) followed by i-PrOH/toluene (1:1, 80 ml/80 ml, 8x/8x). The resulting mixture was heated to clear (78° C.) for 1 hour, slowly cooled to 0° C., and stirred at 0° C. for another 1 hour. The solids were collected by filtration and the cake was washed with 20 ml of i-PrOH/toluene (1:3). The cake was dried under vacuum to constant weight to provide the tert-butylamine salt of 8 as a pale yellow solid (9.7 g, 82% yield, 96% pure, 3.3% A, 0.6% B).

To a 3-necked 250 ml RB flask was added the tert-butylamine salt of 8 (20.0 g, 42.9 mmol) and followed by H₂O/THF/toluene (400 ml/200 ml/160 ml, 20x/10x/8x). The resulting mixture was heated to 60° C. and slowly added 6M HCl until pH of the mixture reached 4.0. The mixture was cooled to room temperature and the aqueous layer was separated. The organic layer was washed with H₂O (100 ml, 5x) and concentrated by rotary evaporating to around 160 ml of overall volume. The solids were collected by filtration and the cake was washed with 20 ml of toluene. The cake was dried under vacuum to constant weight to provide 8 as a pale yellow solid (15 g, 88% yield, 96% pure, 3.3% A, 0.5% B).

Method 3. To a 3-necked 3 L RB flask was added the aqueous solution of the potassium salt containing ˜50 g 8 (90%, 6% A, 4% B, all normalized AUC) and followed by THF/toluene (500 ml/400 ml, 10x/8x). The resulting mixture was heated to 60° C. and slowly added 6M HCl until pH of the mixture reached 4.0. The mixture was cooled to room temperature and the aqueous layer was separated. The organic layer was washed with H₂O (250 ml, 5x) and concentrated by rotary evaporating to around 400 ml of overall volume to afford a slurry of 8 in ˜8x toluene.

To a 3-necked 3 L RB flask was added the slurry (in 8x toluene, 400 ml) and tert-butylamine (67 ml, 5.0 equiv) followed by i-PrOH (400 ml, 8x). The resulting mixture was heated at 78° C. for 1 hour, cooled to 0° C., and stirred at 0° C. for another 1 hour. The solids were collected by filtration and the cake was washed with 100 ml of i-PrOH/toluene (1:3). The cake was dried under vacuum to constant weight to provide the tert-butylamine salt of 8 as a pale yellow solid (42.4 g, 72% yield, 95% pure, 3.2% A, 1.9% B).

To a 3-necked 250 ml RB flask was added the tert-butylamine salt of 8 (42.4 g, 91.0 mmol) and followed by H₂O/THF/toluene (1000 ml/500 ml/400 ml, 20x/10x/8x). The resulting mixture was heated to 60° C. and slowly added 6M HCl until pH reached 4.0. The mixture was cooled to room temperature. The organic layer was separated and washed with H₂O (250 ml, 5x). The organic solution was concentrated by rotary evaporating to ˜400 ml of overall volume. The solids were collected by filtration and the cake was washed with 100 ml of toluene. The cake was dried under vacuum to constant weight to provide 8 as a pale yellow solid (35.4 g, 89.5% yield, 96% pure, 2.9% A, 1.6% B).

Method 4. To a test tube was added 8 (198.6 mg, 0.5 mmol) and cinchonidine (167.1 mg) followed by acetonitrile (7.5 ml). The resulting mixture was heated to clear and cooled to room temperature, and stirred for another 2 hours. The solids were collected by filtration and the cake was washed with 1 ml of MTBE. The cake was dried under vacuum to constant weight to provide the final product (208 mg, 68% yield, 92% pure, 4.4% A, 1.4% B).

### 6.14. Preparation of (S)-3-(4-(2-Amino-6-chloropyrimidin-4-yl)phenyl)-2-(tert-butoxycarbonylamino)propanoic acid (8) using potassium Carbonate as Base

To a 500 ml 3-neck round-bottom flask equipped with a mechanical stirrer, a thermocontroller was charged 2-amino-4,6-dichloropyrimidine (12.57 g, 1.5 equiv), boronate compound 7 (20.00 g, 51.1 mmol), potassium carbonate (21.19 g, 3.0 equiv) and ethanol/water (200 ml, 5:1 by volume). The mixture was stirred and the catalyst bis(triphenylphosphine)palladium(II) dichloride (359 mg, 1 mol %) was added. The mixture was heated to 80° C. and stirred for 2 hours. The reaction was cooled to room temperature and diluted with water (100 ml). The mixture was then concentrated under reduced pressure to remove most of ethanol and 1 N NaOH (60 ml) was added. The mixture was extracted twice with ethyl acetate (2×200 ml) and the aqueous layer was acidified to pH ˜3 using 1 N HCl. The mixture was extracted with ethyl acetate twice (200 ml and 100 ml, respectively) and the combined organic layers were concentrated and the residue was purified by column chromatography (gradient 1:20 to 1:10 methanol/dichloromethane) to afford compound 8 as a pale yellow solid (15.92 g, 79%).

### 6.15. Preparation of (S)-3-(4-(2-Amino-6-chloropyrimidin-4-yl)phenyl)-2-(tert-butoxycarbonylamino)propanoic Acid Using the Lithium Salt of (S)-2-(tert-butoxycarbonylamino)-3-(4-(4,4,5,5-tetramethyl-1,3,2-dioxaborolan-2-yl)phenyl)propanoic Acid

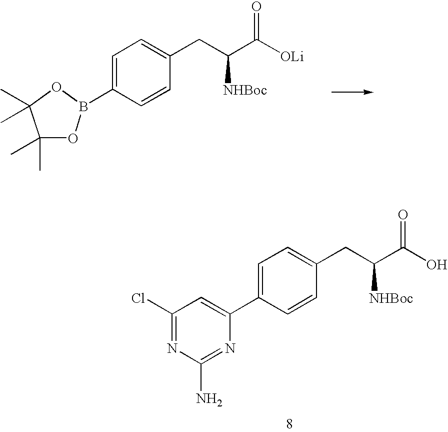

During preparation of compound 7, the isolation of the free acid can be optionally omitted. Thus, an aqueous solution of the lithium salt of compound 7 in 100 ml water, prepared from 5.0 g of Boc-Tyr-OMe (4, 17 mmol), was mixed 2-amino-4,6-dichloropyrimidine (3.3 g, 1.2 eq), potassium bicarbonate (5.0 g, 3 eq), bis(triphenylphosphine)palladium(II) dichloride (60 mg, 0.5 mol %), and 100 ml ethanol. The resulting mixture was heated at 70° C. for 5 hours. Additional 2-amino-4,6-dichloropyrimidine (1.1 g, 0.4 eq) was added and heating was continued at 70° C. for an additional 2 hours. HPLC analysis showed about 94% conversion. Upon cooling and filtration, the filtrate was analyzed by HPLC against a standard solution of compound 8. The assay indicated 3.9 g compound 8 was contained in the solution (59% yield from compound 4).

### 6.16. Alternative Procedure for Preparation of (S)-3-(4-(2-Amino-6-chloropyrimidin-4-yl)phenyl)-2-(tert-butoxycarbonylamino)propanoic Acid Using Potassium Carbonate as Base

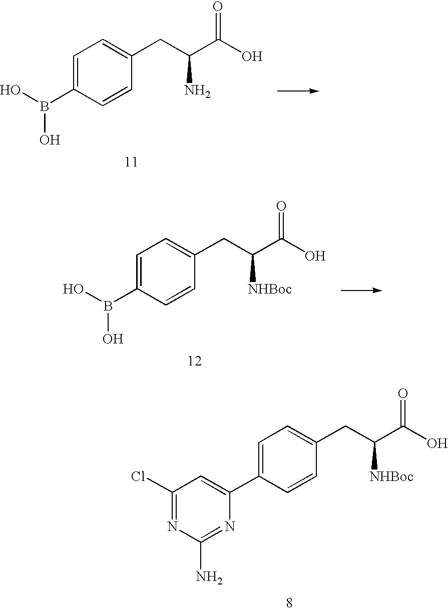

The boronic acid compound 11 (Ryscor Science, Inc., North Carolina, 1.0 g, 4.8 mmol) and potassium carbonate (1.32 g, 2 eq) were mixed in aqueous ethanol (15 ml ethanol and 8 ml water). Di-tert-butyldicarbonate (1.25 g, 1.2 eq) was added in one portion. After 30 minutes agitation at room temperature, HPLC analysis showed complete consumption of the starting compound 11. The 2-amino-4,6-dichloropyrimidine (1.18 g, 1.5 eq) and the catalyst bis(triphenylphosphine)palladium(II) dichloride (34 mg, 1 mol %) were added and the resulting mixture was heated at 65-70° C. for 3 hours. HPLC analysis showed complete consumption of compound 12. After concentration and filtration, HPLC analysis of the resulting aqueous solution against a standard solution of compound 8 showed 1.26 g compound 8 (67% yield).

### 6.17. Alternative procedure for preparation of (S)-3-(4-(2-Amino-6-chloropyrimidin-4-yl)phenyl)-2-(tert-butoxycarbonylamino)propanoic Acid Using Potassium Carbonate/Potassium Bicarbonate as Base

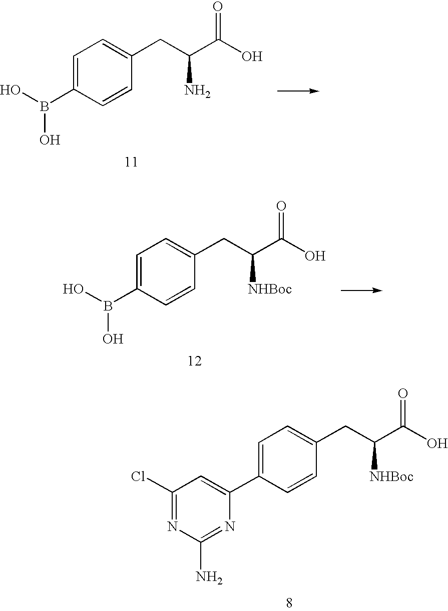

The boronic acid compound 11 (10 g, 48 mmol) and potassium bicarbonate (14.4 g, 3 eq) were mixed in aqueous ethanol (250 ml ethanol and 50 ml water). Di-tert-butyldicarbonate (12.5 g, 1.2 eq) was added in one portion. HPLC analysis indicated that the reaction was not complete after overnight stirring at room temperature. Potassium carbonate (6.6 g, 1.0 eq) and additional di-tert-butyldicarbonate (3.1 g, 0.3 eq) were added. After 2.5 hours agitation at room temperature, HPLC analysis showed complete consumption of the starting compound 11. The 2-amino-4,6-dichloropyrimidine (11.8 g, 1.5 eq) and the catalyst bis(triphenylphosphine)-palladium(II) dichloride (0.34 g, 1 mol %) were added and the resulting mixture was heated at 75-80° C. for 2 hours. HPLC analysis showed complete consumption of compound 12. The mixture was concentrated under reduced pressure and filtered. The filtrate was washed with ethyl acetate (200 ml) and diluted with 3:1 THF/MTBE (120 ml). This mixture was acidified to pH about 2.4 by 6 N hydrochloric acid. The organic layer was washed with brine and concentrated under reduced pressure. The residue was precipitated in isopropanol, filtered, and dried at 50° C. under vacuum to give compound 8 as an off-white solid (9.0 g, 48% yield). Purity: 92.9% by HPLC analysis. Concentration of the mother liquor yielded and additional 2.2 g off-white powder (12% yield). Purity: 93.6% by HPLC analysis.

### 6.18. Preparation of (S)-3-(4-(2-Amino-6-((R)-2,2,2-trifluoro-1-(3′-methoxybiphenyl-4-yl)ethoxy)pyrimidin-4-yl)phenyl)-2-(tert-butoxycarbonylamino)propanoic Acid

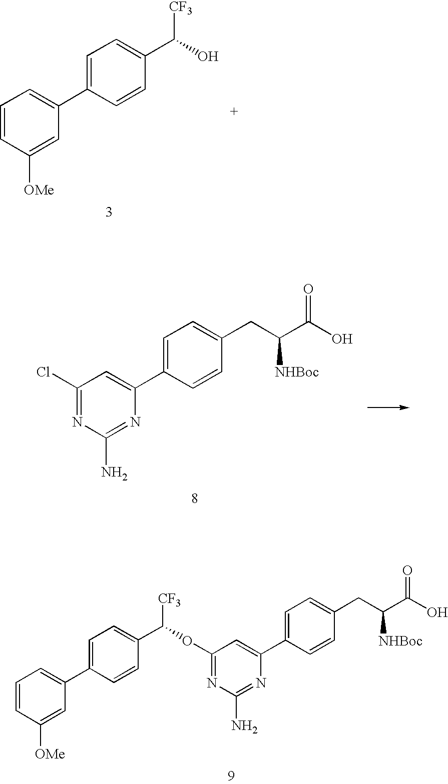

To a 250 ml 3-neck round-bottom flask equipped with a mechanical stirrer, a thermocontroller was charged monochloride 8 (20.39 g, 51.9 mmol), alcohol 3 (17.58 g, 1.2 equiv), cesium carbonate (84.55, 5.0 equiv) and dioxane (205 ml). The mixture was heated to 100° C. and stirred for 17 hours. The reaction was cooled to room temperature and diluted with water (80 ml). Two phases were split and the organic layer was collected and diluted with ethyl acetate (200 ml), washed with a mixture of brine (50 ml) and 1 N HCl (50 ml). The organic layer was concentrated and the residue was purified by column chromatography (gradient: 1:30 to 1:20 methanol/dichloromethane and 0.5% acetic acid) to afford compound 9 as a yellow solid. This solid was recrystallized from EtOH and heptane to give 21.78 g pale yellow solid. Further crystallization of the mother liquor gave 2.00 g pale yellow solid (overall 23.78 g, 72% yield). Chiral analysis of the corresponding methyl ester derivative, prepared using trimethylsilyldiazomethane, showed no detectable amount of the diastereomers. LC-MS (ESI): MH⁺=639.2. ¹H NMR (DMSO-d₆) δ 12.60 (br s, 1H), 8.00 (d, J=8.0 Hz, 2H), 7.77 (d, J=8.0 Hz, 2H), 7.67 (d, J=8.0 Hz, 2H), 7.37 (m, 3H), 7.21 (m, 2H), 7.13 (d, J=8.0 Hz, 1H), 6.96 (m, 1H), 6.84 (m, 2H), 6.75 (s, 2H, 4.15 (m, 1H), 3.82 (s, 3H), 3.10 (dd, J=13.6, 4.4 Hz, 1H), 2.89 (dd, J=13.6, 10.4 Hz, 1H), 1.32 (s, 9H). ¹³C NMR (DMSO-d₆) δ 173.4, 168.4, 166.1, 162.9, 159.7, 155.4, 141.5, 140.8, 134.8, 130.7, 130.0, 129.3, 128.4, 127.2, 126.6, 124.1 (q, J=281 Hz), 119.1, 113.4, 112.3, 91.3, 78.0, 71.3 (q, J=30 Hz), 55.1, 54.9, 36.2, 28.1. ¹⁹F NMR (DMSO-d₆): δ −74.6 (d, J=7.2 Hz). Anal. Calcd. for C₃₃H₃₃F₃N₄O₆: C, 62.06; H, 5.21; N, 8.77. Found: C, 62.25; H, 5.10; N, 8.69.

### 6.19. Preparation of (S)-2-Amino-3-(4-(2-amino-6-((R)-2,2,2-trifluoro-1-(3′-methoxybiphenyl-4-yl)ethoxy)pyrimidin-4-yl)phenyl)propanoic Acid

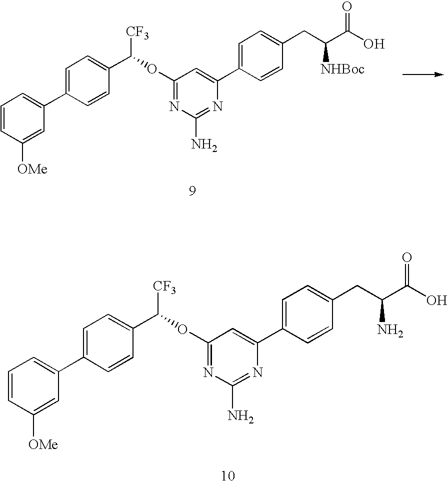

To a 500 ml round-bottom flask was added compound 9 (20.00 g, 31.32 mmol) and THF (100 ml). The solid was dissolved upon stirring and 6 N hydrochloric acid (100 ml) was added slowly. The mixture was then stirred at room temperature for 14 hours. The reaction was diluted with water (100 ml) and most of THF was removed under reduce pressure. The resulting aqueous solution was then transferred to a 500 ml three-necked round-bottom flask equipped with a mechanical stirrer, a pH meter, a thermocontroller and an addition funnel. At 60° C., a solution of 50% aqueous sodium hydroxide was added slowly until pH=4, then a solution of 1 N aqueous sodium hydroxide was added until pH reached 6.5. The mixture was stirred at 60° C. for additional 30 minutes and the solid was collected by filtration and oven-dried under vacuum to give compound 10 (16.30 g, 96% yield) as a pale yellow solid. LC-MS (ESI): MH⁺=539.1. ¹H NMR (DMSO-d₆) δ 8.01 (d, J=8.0 Hz, 2H), 7.76 (d, J=8.0 Hz, 2H), 7.67 (d, J=8.0 Hz, 2H), 7.38 (m, 3H), 7.23 (m, 2H), 6.96 (d, J=8.0 Hz, 1H), 6.81 (m, 3H), 3.81 (s, 3H), 3.59 (br m, 1H), 3.00 (br m, 1H). ¹³C NMR (DMSO-d₆) 169.9, 168.4, 166.1, 162.9, 159.7, 141.5, 140.8, 140.8, 140.0, 134.9, 130.7, 130.0, 129.7, 128.4, 127.2, 126.8, 124.1 (q, J=281 Hz), 119.1, 113.4, 112.3, 91.2, 71.4 (q, J=30 Hz), 55.1, 55.0, 36.5. ¹⁹F NMR (DMSO-d₆): δ −74.6 (d, J=6.8 Hz).

### 6.20. One-pot Preparation of (S)-2-Amino-3-(4-(2-amino-6-((R)-2,2,2-trifluoro-1-(3′-methoxybiphenyl-4-yl)ethoxy)pyrimidin-4-yl)phenyl)propanoic Acid

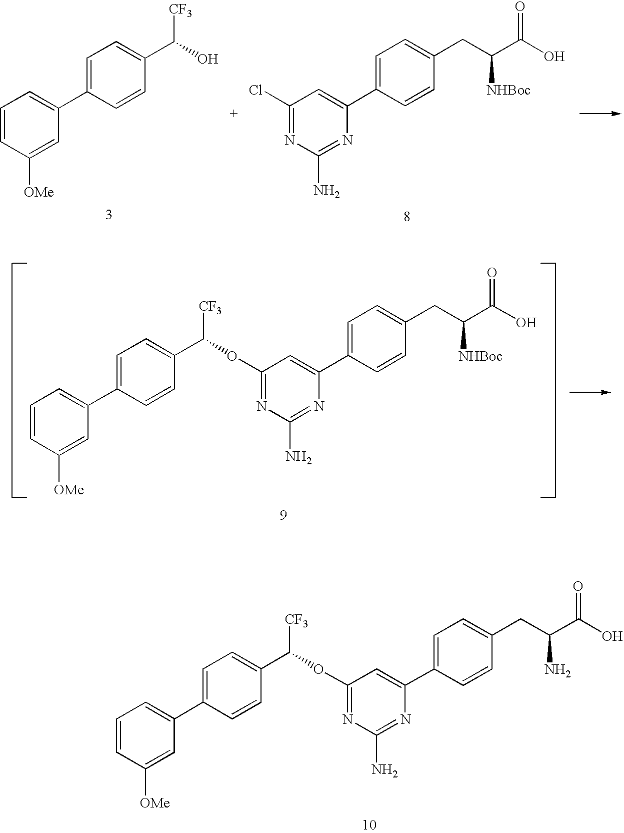

To a 3-neck 250 ml round-bottom flask equipped with a mechanical stirrer, a thermocontroller, was charged compound 3 (8.62 g, 1.2 equiv), 8 (10.00 g, 25.46 mmol), tetrabutylammonium bisulfate (0.86 g, 10 mol %), and cesium carbonate (29.04 g, 3.5 equiv). Dioxane (50 ml) was added and the resulting mixture was heated at 100° C. for 18 hours. HPLC analysis showed 99% conversion of the starting material 8. The mixture was cooled down to 60° C. and water (60 ml) was added. The top organic layer was diluted with THF (80 ml), washed with brine (50 ml), transferred to a 500 ml round-bottom flask, and 80 ml of 6 N hydrochloric acid was added. The mixture was stirred at room temperature for 16 hours. LC-MS analysis of the reaction mixture showed complete consumption of the intermediate compound 9. The reaction mixture was transferred to a 500 ml separatory funnel. The round-bottom flask was washed with water (2×40 ml) and the washes were also transferred to the funnel. The mixture was washed with ethyl acetate (2×100 ml) and the aqueous layer was collected and concentrated at 40° C. (bath temperature) under 80 mbar vacuum to remove any remaining organic solvents. The resulting aqueous solution was then transferred to a 500 ml three-necked round-bottom flask equipped with a mechanical stirrer, a pH meter, a thermocontroller and an addition funnel. At 60° C., a solution of 50% aqueous sodium hydroxide solution was added slowly until pH=4, then a solution of 1N aqueous sodium hydroxide was added until pH reached 6.5. The mixture was stirred at 60° C. for additional 30 minutes and the yellow solids were collected by filtration. HPLC analysis of this solid showed a purity of about 95%. The solids were dried under vacuum at 50° C. overnight to give the crude product compound 10 as a yellow solid (9.48 g, 69% overall yield).

The above solids (9.48 g) were transferred to a 500 ml round-bottom flask and water (95 ml) was added. The mixture was heated at 80° C. (bath temperature) and THF (40 ml) was added dissolve the solids. Most of THF was then removed under vacuum at 80° C. The precipitate was added acetonitrile (80 ml) and was stirred at 80° C. for 2 hours, cooled down to room temperature and then stirred at 0° C. for 30 minutes. The solid was collected by filtration, washed with water (2×50 ml) to give compound 10 as a pale yellow solid (8.53 g, 90% recovery, 62% overall yield). HPLC analysis showed a purity greater than 99%.

### 6.21. One-pot Preparation of (S)-2-Amino-3-(4-(2-amino-6-((R)-2,2,2-trifluoro-1-(3′-methoxybiphenyl-4-yl)ethoxy)pyrimidin-4-yl)phenyl)propanoic Acid

A 22-L, round-bottom flask equipped with a mechanical stirrer, a thermocouple attached to a temperature controller, and a condenser with a nitrogen line was charged with 1,4-dioxane (4 vol) followed by the addition of Cs₂CO₃ (2.03 kg, 3.5 equiv), compound 3 (603 g, 1.2 equiv) and tetrabutylammonium bisulfate (102.8 g, 0. 147 wt). The slurry was slowly heated to 70° C. and then a slurry of compound 8 (700.0 g, 1.782 mol, 1 wt) in 1,4-dioxane (1.5 vol) was added in three portions over 10 minutes. The beaker containing 8 was rinsed with 1,4-dioxane (0.5 vol) and added to the reactor. The reaction became thick briefly after stirring for 15-30 minutes but the entire batch was stirrable. The controller was heated at 78° C. overnight followed by heating at 98° C. for 8 h then 85° C. overnight. HPLC analysis indicated that there were 2.1% of 8 remaining. The reaction was quenched at 78° C. with water (6 vol) and then cooled further. At 42° C., the batch was transferred to a separatory funnel and the two phases separated. The organic phase was then diluted with THF (8 vol) and washed with brine (5 vol). The phases were separated and the organic phase was washed with brine (5 vol). The phases were separated and the organic phase (9.5 L) was transferred to a 22-L reactor. A solution of 6 N HCl (11.4 vol) was added and the batch was heated at 40-45° C. for 2 hours. HPLC analysis indicated that the reaction was complete and Darco G-60 (0.33 wt.) and water (2 vol) were added. The batch was stirred at 40° C. over the weekend and then heated to 60° C. The reaction mixture was filtered at 60° C. through PTFE cloth and the reactor was rinsed with water (6 vol). The rinse was heated to 60° C. and washed through the Darco pad. The filtrate was then passed through a 0.3-μm in-line filter and washed with IPAc twice (10 vol, 8.8 vol). The aqueous phase was then concentrated under reduced pressure at 45° C. using a 20-L, rotary evaporator until the mixture turned cloudy (2-3 h). The volume of distillate collected was approximately 3.3 L. The batch was then transferred back to a 22-L reactor and held at 40° C. overnight.

The batch was heated to 60° C. whereupon the batch turned from cloudy to clear. To a separate 22-L reactor was charged water (1.6 vol) and 85% phosphoric acid (0.24 vol) and the pH was adjusted to 6.5 using a 50% NaOH solution (approximately 0.3 vol). The acidic product solution was then transferred via peristaltic pump to the reactor containing the pH 6.5 buffered solution and the pH was maintained within 6 and 7 through the addition of 50% NaOH (approximately 3.5 vol). The temperature of the reactor was maintained between 55 and 65° C. (2-h addition time). Once the addition was complete, the slurry was heated at 60-65° C. for 90 minutes, filtered, and washed with water (2×6.7 vol). The wet cake was dried in a vacuum oven at 55° C. for 39 h to afford 635 g of crude 10 as a yellow solid (66% yield). The purity of the product was 93.2% (AUC).

### 6.22. Purification of (S)-2-Amino-3-(4-(2-amino-6-((R)-2,2,2-trifluoro-1-(3′-methoxybiphenyl-4-yl)ethoxy)pyrimidin-4-yl)phenyl)propanoic Acid

A 22-L, round-bottom flask equipped with a mechanical stirrer, a thermocouple attached to a temperature controller, and a condenser with a nitrogen line was charged with crude 10 (630 g) followed by the addition of THF (5 vol). The slurry was heated to 65° C. After 30 minutes, a solution of 5-6 N HCl in IPA (0.47 L, 0.746 vol) was added and the solids slowly dissolved. The orange solution was heated at 65° C. for 30 minutes IPA (10 vol) was slowly added maintaining the temperature between 60-70° C. Once the addition was complete, the mixture was stirred for 20 minutes and then IPAc (10 vol) was slowly added maintaining the temperature between 60-70° C. Once the addition was complete, the thick slurry was stirred at 65° C. for 1 hour and then cooled to 27° C. over 4.5 hours. The solids were filtered and washed with IPA (2×3 vol). The product was dried in a vacuum oven at 55° C. for 15 hours to afford 630 g of 10 diHCl salt (88% yield) with a purity of 95.0% (AUC).

A 12-L, round-bottom flask equipped with a mechanical stirrer, a thermocouple attached to a temperature controller, and a pH probe attached to a pH meter was charged with 10 diHCl salt (620 g, 1 wt) followed by an aqueous solution of 1 M NaOH (10 vol). The mixture was heated to 40° C., stirred until all the solids dissolved (2 hours), and then transferred to a 10-L carboy. The 12-L, round-bottom flask was washed with water and then 85% phosphoric acid (124 ml, 0.2 vol) and water (1.3 vol) were charged to the reactor. The pH was adjusted to 6.5 using 50% NaOH (0.24 vol) and then heated to 65° C. The product solution in the carboy was transferred via peristaltic pump to the pH buffered solution and the pH was maintained between 6 and 7 through the addition of an aqueous solution of 6 M HCl (0.67 L). Once the addition was complete, the slurry was heated at 65° C. for 3 hours and the solids were filtered. The cake was washed with water (3×5 vol) and then dried in a vacuum oven at 55° C. for 41 hours to afford 473 g of 10 as a light yellow solid (87% yield) with a purity of 97.7% (AUC).

### 6.23. Preparation of Crystalline (S)-2-amino-3-(4-(2-amino-6-((R)-2,2,2-trifluoro-1-(3′-methoxybiphenyl-4-yl)ethoxy)pyrimidin-4-yl)phenyl)propanoate Tosylate Anhydrate

(S)-2-amino-3-(4-(2-amino-6-((R)-2,2,2-trifluoro-1-(3′-methoxybiphenyl-4-yl)ethoxy)pyrimidin-4-yl)phenyl)propanoate free base (11.8 g assay, 22.0 mmol) is added to a solution of TsOH.H₂O (4.60 g, 24.0 mmol) in THF (35 ml) and water (2.8 ml) at 40° C. Acetonitrile (35 ml) is added, and the mixture is aged until a slurry is obtained. More acetonitrile (105 ml) is added slowly over 1 hour. The mixture is aged at 40° C. for 2 hours, and then slowly cooled to 20° C. over 3 hours. After aging at 20° C. for 5 hours, the mixed is filtered and washed with ACN/THF/H₂O (50/10/1 ml). The filter cake is dried in a vacuum oven at 40° C. with slow nitrogen sweep to afford the title compound.

### 6.24. Preparation of Crystalline (S)-2-amino-3-(4-(2-amino-6-((R)-2,2,2-trifluoro-1-(3′-methoxybiphenyl-4-yl)ethoxy)pyrimidin-4-yl)phenyl)propanoate Tosylate Monohydrate

A 500 ml 3-necked RBF was charged with (S)-2-amino-3-(4-(2-amino-6-((R)-2,2,2-trifluoro-1-(3′-methoxybiphenyl-4-yl)ethoxy)pyrimidin-4-yl)phenyl)propanoic acid (zwitterion, 10 g, 18.6 mmol), p-toluenesulfonic acid monohydrate (3.94 g, 20.4 mmol), CH₃CN (50 ml, 5X) and water (10 ml, 1X) at room temperature. The suspension was heated to 75° C. at gentle reflux. Then AcOH (20 ml, 2X) was added to give a clear yellow solution (the first 10 ml AcOH already made the suspension clear). To this clear solution was added CH₃CN (60 ml, 6X), seeding at this point gave a cloudy solution, then additional CH₃CN (40 ml, 4X) was added. The resulting slurry was stirred at 80° C. for 40 minutes, then it was allowed to cool to room temperature with stirring and further stirred overnight and filtered and washed with CH₃CN/water (15/1, 40 ml, 4X). The wet pale yellow cake was dried at 40° C. under vacuum overnight to give a off-white solid (9.3 g, 68.6% recovery, 98.8% pure by released 40 minutes method, KF=0.93%). Upon standing on lab bench over four days with a paper cover, the KF rose to 3.262%, and the weight rose to 9.61 g (71%).

### 6.25. Preparation of Crystalline (S)-2-amino-3-(4-(2-amino-6-((R)-2,2,2-trifluoro-1-(3′-methoxybiphenyl-4-yl)ethoxy)pyrimidin-4-yl)phenyl)propanoate Tosylate Dihydrate

(S)-2-amino-3-(4-(2-amino-6-((R)-2,2,2-trifluoro-1-(3′-methoxybiphenyl-4-yl)ethoxy)pyrimidin-4-yl)phenyl)propanoate (120.0 g, 88 w %, 105.6 g active, 196 mmol) was added to a solution of TsOH.H₂O (39.8 g, 209 mmol) in a mixture of THF (240 ml) and water (48 ml). The mixture was heated to 50° C. to give a homogeneous solution. Approximately 120 ml of a mixture of ACN/water (1200/60 ml) was added and the mixture was seeded with the captioned compound (0.63 g). After aging for 1 hour at 40° C., a nice slurry was obtained. The remaining ACN/water mixture was added slowly over 3 hours at 40° C. and the slurry was aged at 40° C. for 2 hours then slowly cooled to 20° C. and aged overnight. The solid was collected by filtration and the filter cake was washed with 5/1 ACN/THF with ˜5 vol % water (500 ml). Air drying at room temperature overnight gave 138.5 g of the product as a white solid (99.5 A%, 93.4% yield corrected for purity). Loss in the mother liquor and wash was 6.5%. KF of solid was 4.4%.

### 6.26. Preparation of Crystalline (S)-2-amino-3-(4-(2-amino-6-((R)-2,2,2-trifluoro-1-(3′-methoxybiphenyl-4-yl)ethoxy)pyrimidin-4-yl)phenyl)propanoate Maleate

To a solution of (S)-2-amino-3-(4-(2-amino-6-((R)-2,2,2-trifluoro-1-(3′-methoxybiphenyl-4-yl)ethoxy)pyrimidin-4-yl)phenyl)propanoate (13.3 mg) in 1 ml of methanol was added maleic acid (3.2 mg). The mixture was heated to gentle reflux and then cooled to room temperature to yield the title compound.

All references (e.g., patents and patent applications) cited above are incorporated herein by reference in their entireties.

What is claimed is:

1. A crystalline compound, which is (S)-2-amino-3-(4-(2-amino-6-((R)-2,2,2-trifluoro-1-(3′-methoxybiphenyl-4-yl)ethoxy)pyrimidin-4-yl)phenyl)propanoic acid or a salt thereof.

2. A crystalline compound, which is (S)-2-amino-3-(4-(2-amino-6-((R)-2,2,2-trifluoro-1-(3′-methoxybiphenyl-4-yl)ethoxy)pyrimidin-4-yl)phenyl)propanoate tosylate.

3. The crystalline compound of claim 2, which is anhydrous.

4. The crystalline compound of claim 3, which has a melting point of about 241° C.

5. The crystalline compound of claim 3, which has an X-ray powder diffraction pattern comprising a peak at one or more of about 3.5, 7.0, 8.6, 10.9, 13.5, 14.0, 15.1, 17.3 and/or 20.5 degrees 2θ.

6. The compound of claim 3, which has an X-ray powder diffraction pattern substantially the same as that shown in FIG. 1.

7. The crystalline compound of claim 2, which is a hydrate.

8. The crystalline compound of claim 7, which is a monohydrate.

9. The crystalline compound of claim 8, which has a melting point of about 221° C.

10. The compound of claim 8, which has an X-ray powder diffraction pattern comprising a peak at one or more of about 3.6, 8.2, 8.7, 13.1, 14.5, 17.5, 18.0, 19.9 and/or 21.4 degrees 2θ.

11. The compound of claim 8, which has an X-ray powder diffraction pattern substantially the same as that shown in FIG. 2.

12. The compound of claim 8, which has a Raman spectrum substantially the same as that shown in FIG. 3.

13. The crystalline compound of claim 7, which is a dihydrate.

14. The crystalline compound of claim 13, which has a melting point of about 238° C.

15. The crystalline compound of claim 13, which has an X-ray powder diffraction pattern comprising a peak at one or more of about 8.6, 9.0, 17.2, 17.8, 18.6, 21.6, 25.2 and/or 26.9 degrees 2θ.

16. The compound of claim 13, which has an X-ray powder diffraction pattern substantially the same as that shown in FIG. 4.

17. A crystalline compound, which is (S)-2-amino-3-(4-(2-amino-6-((R)-2,2,2-trifluoro-1-(3′-methoxybiphenyl-4-yl)ethoxy)pyrimidin-4-yl)phenyl)propanoate maleate.

18. A pharmaceutical dosage form comprising the crystalline compound of claim 1.

19. The pharmaceutical dosage form of claim 18, wherein the crystalline compound is (S)-2-amino-3-(4-(2-amino-6-((R)-2,2,2-trifluoro-1-(3′-methoxybiphenyl-4-yl)ethoxy)pyrimidin-4-yl)phenyl)propanoate tosylate.

20. The pharmaceutical dosage form of claim 18, wherein the crystalline compound is (S)-2-amino-3-(4-(2-amino-6-((R)-2,2,2-trifluoro-1-(3′-methoxybiphenyl-4-yl)ethoxy)pyrimidin-4-yl)phenyl)propanoate maleate.

21. A method of preparing a crystalline salt of (S)-2-amino-3-(4-(2-amino-6-((R)-2,2,2-trifluoro-1-(3′-methoxybiphenyl-4-yl)ethoxy)pyrimidin-4-yl)phenyl)propanoic acid, which comprises:
heating a solution comprising (S)-2-amino-3-(4-(2-amino-6-((R)-2,2,2-trifluoro-1-(3′-methoxybiphenyl-4-yl)ethoxy)pyrimidin-4-yl)phenyl)propanoic acid and a pharmaceutically acceptable acid to provide a salt of (S)-2-amino-3-(4-(2-amino-6-((R)-2,2,2-trifluoro-1-(3′-methoxybiphenyl-4-yl)ethoxy)pyrimidin-4-yl)phenyl)propanoic acid;
reducing the solubility of the salt in the solution under conditions sufficient to provide a crystalline salt of (S)-2-amino-3-(4-(2-amino-6-((R)-2,2,2-trifluoro-1-(3′-methoxybiphenyl-4-yl)ethoxy)pyrimidin-4-yl)phenyl)propanoic acid; and
isolating the crystalline salt.

22. The method of claim 21, wherein the solution comprises THF and water.

23. The method of claim 21, wherein the pharmaceutically acceptable acid is p-toluenesulfonic acid or maleic acid.

24. The method of claim 21, wherein the solubility of the salt is reduced by adding antisolvent and cooling the solution.

25. The method of 24, wherein the anti-solvent is acetonitrile.

26. A method of preparing crystalline (S)-2-amino-3-(4-(2-amino-6-((R)-2,2,2-trifluoro-1-(3′-methoxybiphenyl-4-yl)ethoxy)pyrimidin-4-yl)phenyl)propanoic acid tosylate, which comprises:
heating a solution comprising water, (S)-2-amino-3-(4-(2-amino-6-((R)-2,2,2-trifluoro-1-(3′-methoxybiphenyl-4-yl)ethoxy)pyrimidin-4-yl)phenyl)propanoic acid, and p-toluenesulfonic acid monohydrate;
adding an anti-solvent to the solution to provide a mixture;
cooling the mixture; and
isolating crystalline (S)-2-amino-3-(4-(2-amino-6-((R)-2,2,2-trifluoro-1-(3′-methoxybiphenyl-4-yl)ethoxy)pyrimidin-4-yl)phenyl)propanoic acid tosylate from the mixture.

27. The method of claim 26, wherein the anti-solvent is acetonitrile.

28. A method of treating, preventing or managing a disease or disorder mediated by peripheral serotonin, which comprises administering to a patient in need of such treatment, prevention or management a therapeutically or prophylactically effective amount of crystalline (S)-2-amino-3-(4-(2-amino-6-((R)-2,2,2-trifluoro-1-(3′-methoxybiphenyl-4-yl)ethoxy)pyrimidin-4-yl)phenyl)propanoic acid or a salt thereof.

29. The method of claim 28, wherein the disease or disorder is carcinoid syndrome.

30. The method of claim 28, wherein the disease or disorder is a gastrointestinal disease or disorder.

31. The method of claim 30, wherein the disease or disorder is irritable bowel syndrome.
